(19) United States
(12) Patent Application Publication
(10) Pub. No.: US 2011/0046727 A1
(43) Pub. Date: Feb. 24, 2011

(54) INTRACORNEAL DIFFRACTIVE LENS

(76) Inventor: Gilbert Cohen, Lyon (FR)

Correspondence Address: CANTOR COLBURN LLP, 20 Church Street, 22nd Floor, Hartford, CT, 06103, US

(21) Appl. No.: 12/668,217

(22) PCT Filed: Jul. 9, 2008
(86) PCT No.: PCT/FR08/01001

§ 371 Date: Sep. 22, 2010

(30) Foreign Application Priority Data
Jul. 9, 2007 (FR) 0704963

(51) Int. Cl. A61F 2/16

(52) U.S. Cl. 623/6.27

(57) ABSTRACT

The invention relates to a lens that can be implanted in the cornea for correcting vision defects, such as presbyopia. It consists of a zonal diffractive lens with phase inversion that comprises an alternation of optically active or “full” annular areas (2) and of optically inactive or empty annular areas (3) which are all concentric. The empty annular areas (3) are filled with an optically inactive “cement” that binds together the “full” annular areas (2) in order to ensure the stability thereof. The cement is a hydrogel pervious to nutrients and oxygen having an optical index close to that of the cornea.

### TECHNICAL FIELD

The present invention relates to diffractive lenses, in particular intracorneal lenses designed to be implanted in the cornea in order to correct vision defects, also called ametropias. More particularly, this invention concerns an intracorneal diffractive lens that can be used for surgical correction of presbyopia.

### BACKGROUND

In the correction of ametropias by refractive surgery, a distinction is made between corneal refractive surgery and intraocular surgery, with corneal surgery presenting fewer complications.

Corneal refractive surgery is presently carried out by modifying the curvature of the anterior surface of the cornea.

More particularly, the correction of presbyopia by corneal surgery is based on pseudo-accommodation, that is to say on the transformation of the cornea in multifocal diopter by modification of the curvature of the cornea. In this type of refractive correction, the optical performance depends on the pupil diameter, and thus on the level of illumination.

In the correction of presbyopia by intraocular surgery, the use of diffractive lenses gives good results, which are independent of the centering of the lens and of the pupil diameter.

Transformation of the cornea into a diffractive lens by sculpturing is not possible. Only the use of an intracorneal diffractive lens would afford the benefits of the optical properties of diffractive lenses and the safety of corneal surgery.

The present obstacles to the use of intracorneal implants, in particular of intracorneal diffractive lenses, especially for the treatment of presbyopia, concern the biocompatibility of these implants and especially their permeability to the flow of nutrients and oxygen within the thickness of the cornea, which permeability is essential to maintain the transparency and the refractive function of the cornea.

Hydrogels with a high water content are certainly permeable to nutrients and to oxygen, but they have an index of optical refraction close to that of the cornea and are therefore without any optical efficacy in the production of intracorneal diffractive lenses.

Documents EP 0420549 A2 and WO 99/07309 show examples of corneal lenses that are made from hydrogels and comprise concentric annular zones arranged in steps. It can be understood from these documents that if one of the two components of the lens is not a permeable hydrogel, it forms a continuous layer constituting a barrier to the flow of nutrients and oxygen.

### BRIEF SUMMARY

The present invention aims to solve the problems set out here by providing an intracorneal diffractive lens which is suitable for the treatment of presbyopia and which is designed in such a way as to permit a good circulation of the flow of nutrients and oxygen within the thickness of the cornea when the lens is implanted, while at the same time being easy to manipulate.

To this end, the invention relates to a zonal diffractive lens with phase reversal and with an alternation of optically active or “full” annular zones and of optically inactive or “empty” annular zones, all of these annular zones being concentric or coaxial, said lens being principally characterized in that the “empty” annular zones are occupied by an optically inactive “cement” that interconnects the “full” annular zones in order to ensure the stability of these “full” annular zones.

More particularly, the diffractive lens according to the invention is designed as an intracorneal lens in which the “cement” of the inactive or “empty” annular zones has a permeability to nutrients and to oxygen that is comparable to the permeability of the corneal tissue, and it has an optical index close to that of the cornea.

The “full” annular zones of such a lens can have a different optical index compared to the “empty” annular zones, such that the optical index of the “full” annular zones is:
greater than that of the “empty” annular zones, or
less than that of the “empty” annular zones.

In a preferred embodiment of the intracorneal diffractive lens forming the subject matter of the invention, the “empty” annular zones are filled by a hydrogel which has a high water content and which is permeable but optically inactive and constitutes the “cement” connecting the “full” annular zones, so as to maintain the concentric or coaxial spatial distribution of these “full” annular zones, and which thus facilitates the manipulation of the lens. The hydrogel serving here as a “cement” that connects the “full” annular zones is in particular a hydrogel whose percentage of water is equal to or greater than 78%.

The “full” annular zones can also be made of a hydrogel whose percentage of water is:
less than 78%, preferably between 50 and 70%; or

greater than 78%, preferably greater than 85%, or are even formed by water.

Thus, the intracorneal diffractive lens forming the subject matter of the invention is characterized by an alternation of “full” concentric rings, which are made of a material chosen for its optical index, and of “empty” rings preferably filled with permeable hydrogel to ensure the cohesion of the assembly, which “empty” zones filled with hydrogel are permeable to nutrients and oxygen, and their regular alternation permits good circulation of the flows within the thickness of the cornea.

The geometry of a zonal diffractive lens of this kind, of which the optically active parts are “full” and concentric annular zones separated by gaps, is justified theoretically by the principle of Fresnel lenses and by the concept of phase reversal (Rayleigh-Wood phase reversal zone plate).

At its center, this lens can comprise a profiled disk made of the same material as the “full” annular zones and surrounded concentrically or coaxially by these “full” annular zones, the central disk constituting an optically active zone in the manner of a first ring with an inner radius of zero. Alternatively, the lens comprises, at its center, an “empty” and therefore optically inactive circular zone, which is surrounded coaxially by the first “full” annular zone.

For production reasons, the “full” annular zones of the lens, and if appropriate the central disk, can be connected by a fine membrane made of the same optically active material, said membrane remaining permeable to nutrients because of its very slight thickness.

In another embodiment, and again for production reasons, the “full” annular zones of the lens, and if appropriate the central disk, are connected by material bridges whose general orientation is radial and which are made of the same optically active material, said material bridges extending across the “empty” annular zones.

The membrane or the material bridges facilitate in particular the manipulation of the rings and/or serve as injection channels during production, without modifying the optical properties of the lens or forming an obstacle to the transfer of nutrients.

The intracorneal diffractive lens forming the subject matter of the invention can be produced as a monofocal lens designed to correct spherical ametropias, or as a bifocal lens, this latter version being designed to correct presbyopia. The precise definition of the geometry of the anterior and posterior faces of the “full” annular zones of such a lens contributes to its adaptation to each particular case of use. Moreover, insofar as the “full” annular zones are not only connected by the parts made of permeable hydrogel but are also coated by parts which are made of the same permeable hydrogel and whose anterior and posterior surfaces may or may not be parallel, these parts made of hydrogel may or may not have an additional refractive effect.

### BRIEF DESCRIPTION OF THE DRAWINGS

The invention will be better understood, and other features thereof will become clear, from the following description in which reference is made to the attached schematic drawing showing, by way of example, several embodiments of this intracorneal diffractive lens.

FIG. 1 is a diametric sectional view of an intracorneal diffractive lens according to the present invention, in a first embodiment;

FIG. 2 is a diametric sectional view of an intracorneal diffractive lens according to the present invention, in a second embodiment;

FIG. 3 is a view similar to FIG. 1 and illustrates a variant of the lens according to the first embodiment;

FIG. 4 is a view similar to FIG. 2 and illustrates a variant of the lens according to the second embodiment;

FIG. 5 is a front view of an intracorneal diffractive lens according to a final embodiment of the invention.

### DETAILED DESCRIPTION

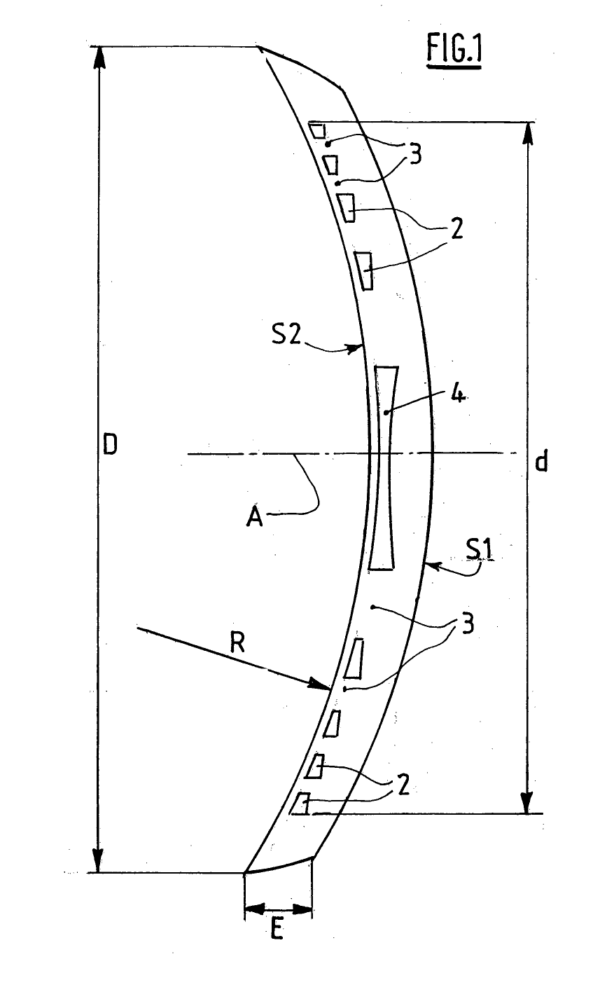

Referring to FIG. 1, an intracorneal diffractive lens whose central axis is designated by A has an external diameter D that can be between 5 and 9 mm, and a mean curvature defined by a radius R that can be between 7 and 9 mm. This lens has a convex outer surface S1 and a concave inner surface S2, and its thickness E measured between the two surfaces S1 and S2 can be between 0.05 mm and 0.5 mm.

The useful zone of the lens, centered on the axis A, is a circle whose diameter d can be between 3 and 7 mm, depending on the external diameter D of this lens. This useful zone comprises a succession of “full” rings 2 made of optically active material and with increasing diameters, all of them being centered on the axis A and being separated from one another by “empty” intermediate annular zones 3. The “full” rings 2 and the “empty” intermediate zones 3 have a width that decreases uniformly from the central axis A in the direction of the periphery of the lens, the geometry of the “full” rings 2 conforming to the principle of the Fresnel zone lens. In the embodiment in FIG. 1, the intracorneal lens further comprises, at its center, a profiled disk 4 which is made of the same optically active material as the “full” rings 2 and which is surrounded concentrically or coaxially by these “full” rings 2. The central disk 4 can be likened to a first “full” ring with inner radius equal to zero.

The “empty” intermediate zones 3 are in fact filled by an optically inactive or weakly active material, which is in particular a hydrogel whose percentage of water is equal to or greater than 78%. This can be a hydrogel of acrylate or methacrylate, acrylamide or methyacrylamide, polyester, vinyl copolymer or similar. This hydrogel is not only present between the “full” rings 2 but can also entirely coat these “full” rings 2, and also the central disk 4, by extending as far as the outer surface S1 and inner surface S2. In all cases, this hydrogel forms a “cement” interconnecting all the rings 2, thereby stabilizing the structure of the lens.

The “full” rings 2 and the central disk 4 are made of a material having an optical index different than that of the cornea. The material can also be a hydrogel, but one whose percentage of water is less than 78%, preferably between 50% and 70%.

The “full” rings 2, which can be between five and thirty in number (the drawing shows in a simplified way a very small number of rings), have a permeability less than that of the cornea and provide, together with the disk 4, the diffraction needed for the desired correction of vision.

The “empty” intermediate annular zones 3, filled with hydrogel, provide the connection between the rings 2 while being permeable to the flow of nutrients and to oxygen.

The outer surface S1 and inner surface S2 can be parallel and thus have no effect on the correction that is obtained, or, by contrast, they can be non-parallel and configured in such a way as to participate in the visual correction by virtue of an additional refractive effect.

Such an intracorneal diffractive lens combining two materials can be produced by molding and overmolding techniques. In particular, it can be produced by a twin injection procedure.

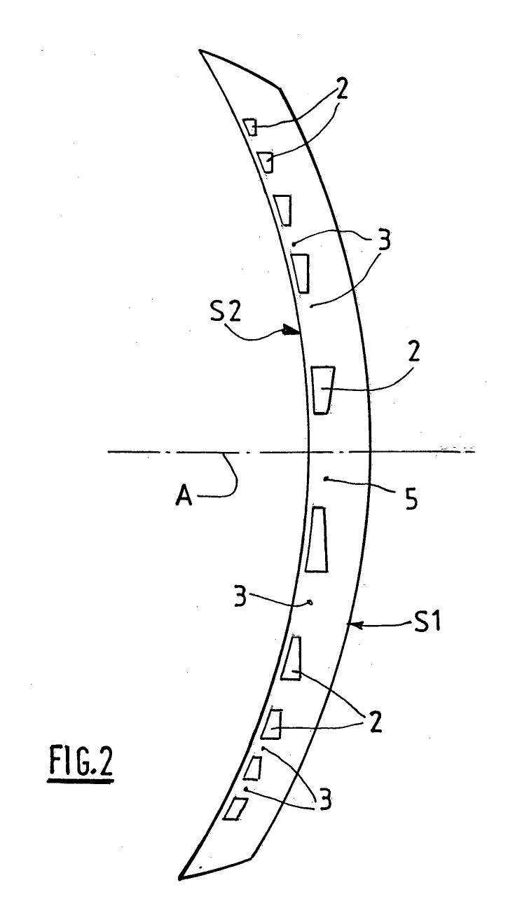

FIG. 2, in which the elements corresponding to those described above are designated by the same reference signs (letters or numbers), shows a variant of this intracorneal diffractive lens. In this variant, the central disk is omitted. The lens thus comprises, at its center, an “empty” circular zone 5, or one that is filled with optically inactive or weakly active but permeable material, such as a suitable hydrogel; the central circular zone 5 is surrounded concentrically by the first “full” annular zone, that is to say by the first ring 2.

In one variant, not illustrated directly in the drawing, of this intracorneal diffractive lens, the rings 2 have a lower optical index than that of the “cement” that connects these rings. In this case, the “cement” remains in particular a hydrogel whose water content is close to 78%, while the rings 2 are made of a hydrogel whose water content is greater than that of said “cement” and is typically greater than 85% or are even formed by water.

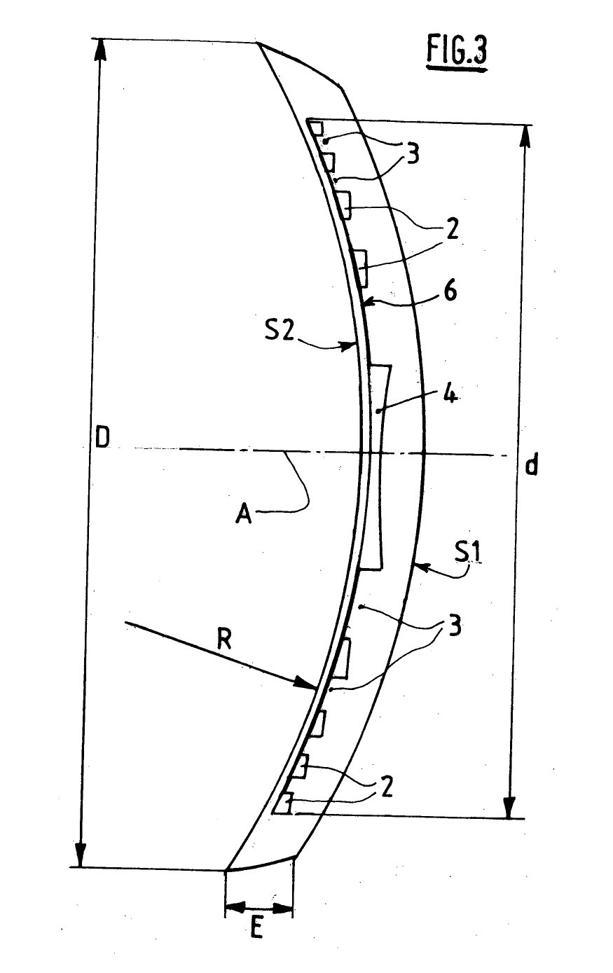

FIG. 3 illustrates a variant, of the lens according to FIG. 1, in which the “full” rings 2 and the central disk 4 are interconnected by a fine membrane 6 made of the same optically active material. The membrane 6, here embedded in the “empty” intermediate annular zones 3, remains permeable to nutrients because of its very slight thickness, and it thus still allows said “empty” annular zones 3 to perform their function.

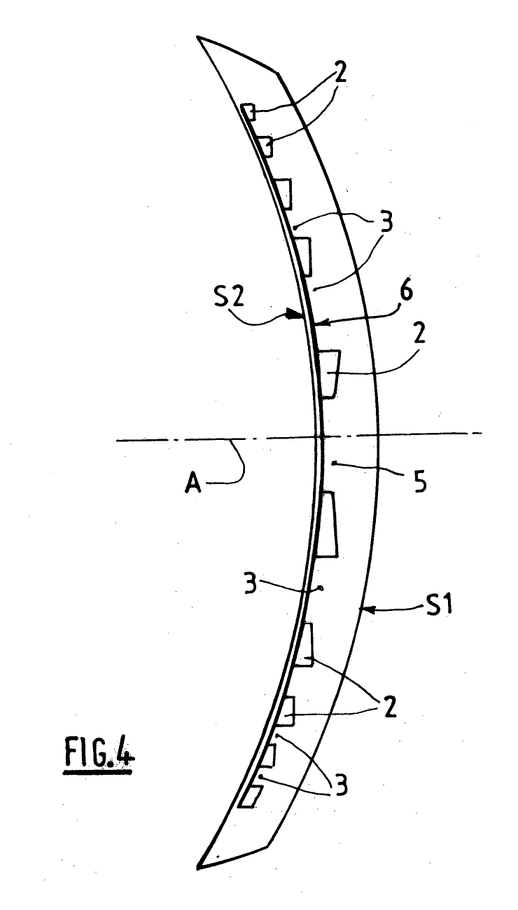

FIG. 4 similarly illustrates a variant, of the lens according to FIG. 2, in which the “full” rings 2 are interconnected by a fine membrane 6 made of the same optically active material. In the absence of a central disk, the membrane 6 is here present in the “empty” intermediate annular zones 3 and also in the central circular zone 5 and, as before, it does not form an obstacle to the transfer of nutrients.

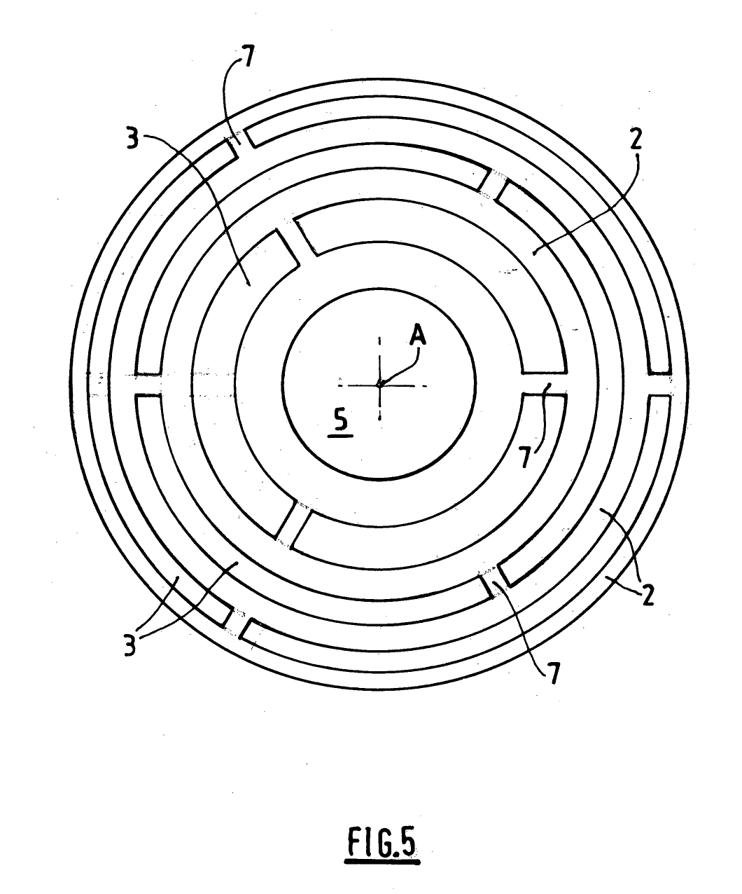

Finally, FIG. 5 shows another embodiment, in which the “full” rings 2 of the lens are interconnected by material bridges 7 of radial orientation that are made of the same optically active material as these rings 2. By virtue of being thin, the material bridges 7 form, between themselves and the rings 2, wide spaces in the shapes of arcs of a circle that are filled by the inactive but permeable material of the “empty” intermediate zones 3.

As will be appreciated, the presence of the membrane 6 or of the material bridges 7 facilitates the production of the lens, without impairing vision and without adversely affecting the permeability of the “empty” intermediate annular zones 3.

It would not constitute a departure from the scope of the invention, as defined in the attached claims, if one were to modify:
the dimensions of the lens;
the nature of its component materials;
the number of its full rings;
the nature of the sight defect corrected by this lens.

1. A zonal diffractive lens with phase reversal and with an alternation of optically active or “full” annular zones and of optically inactive or “empty” annular zones, all of these annular zones being concentric or coaxial, wherein the “empty” annular zones are occupied by an optically inactive “cement” that interconnects the “full” annular zones in order to ensure stability of these “full” annular zones.

2. The diffractive lens as claimed in claim 1, designed as an intracorneal lens, wherein the “cement” of the inactive or “empty” annular zones has a permeability to nutrients and to oxygen that is comparable to a permeability of corneal tissue and has an optical index close to that of a cornea.

3. The intracorneal diffractive lens as claimed in claim 2, wherein the “full” annular zones have a different optical index compared to the empty annular zones, such that the optical index of the full annular zones is greater than that of the empty annular zones.

4. The intracorneal diffractive lens as claimed in claim 2, wherein the “full” annular zones have a different optical index compared to the empty annular zones, such that the optical index of the full annular zones is less than that of the empty annular zones.

5. The intracorneal diffractive lens as claimed in claim 3, wherein the “empty” annular zones are filled by a hydrogel which has a high water content and which is permeable but optically inactive and constitutes the “cement” interconnecting the “full” annular zones.

6. The intracorneal diffractive lens as claimed in claim 5, wherein the hydrogel that serves as a “cement” connecting the “full” annular zones is a hydrogel having a percentage of water equal to or greater than 78%.

7. The intracorneal diffractive lens as claimed in claim 6, wherein the “full” annular zones are also made of a hydrogel whose percentage of water is less than 78%.

8. The intracorneal diffractive lens as claimed in claim 6, wherein the “full” annular zones are also made of a hydrogel whose percentage of water is greater than 78%.

9. The intracorneal diffractive lens as claimed in claim 2, wherein the “full” annular zones are not only connected by the parts made of permeable hydrogel but are also coated by parts made of the same hydrogel.

10. The intracorneal diffractive lens as claimed in claim 1, wherein it comprises, at a center, a profiled disk made of the same active material as the “full” annular zones and surrounded concentrically or coaxially by these “full” annular zones, the central disk constituting an optically active zone comprising a first ring with an inner radius of zero.

11. The intracorneal diffractive lens as claimed in claim 1, wherein it comprises, at a center, an “empty” and therefore optically inactive circular zone, which is surrounded concentrically by the first “full” annular zone.

12. The intracorneal diffractive lens as claimed in claim 10, wherein the “full” annular zones, and if appropriate the central disk, are connected by a fine membrane made of the same optically active material, said membrane remaining permeable to nutrients because of its very slight thickness.

13. The intracorneal diffractive lens as claimed in claim 10, wherein the “full” annular zones, and if appropriate the central disk, are connected by material bridges whose general orientation is radial and which are made of the same optically active material, said material bridges extending across the “empty” annular zones.

14. The intracorneal diffractive lens as claimed in claim 1, wherein it is produced as a bifocal lens designed to correct presbyopia.
